(19) United States
(12) Patent Application Publication
(10) Pub. No.: US 2004/0224881 A1
(43) Pub. Date: Nov. 11, 2004

(54) Method for screening compounds for activity in treating an osteoclast related bone disease

(76) Inventors: Anne Marie Heegaard, Herlev (DK); Michael T. Engsig, Herlev (DK); Lars S. Madsen, Ballerup (DK); Bo S. Jensen, Ballerup (DK); Palle Christophersen, Ballerup (DK); Martin Stahlhut, Herlev (DK); Morten A. Karsdal, Herlev (DK)

Correspondence Address: Edwards & Angell, LLP, Intellectual Property Practice Group, P.O. Box 55874, Boston, MA, 02205, US

(21) Appl. No.: 10/623,150

(22) Filed: Jul. 18, 2003

Related U.S. Application Data
(63) Continuation of application No. PCT/DK02/00037, filed on Jan. 17, 2002.
(60) Provisional application No. 60/265,874, filed on Feb. 5, 2001.

(30) Foreign Application Priority Data
Jan. 23, 2001 (DK) PA 2001 00118

(51) Int. Cl. C12Q 1/00; A61K 38/17

(52) U.S. Cl. 514/12; 435/4

(57) ABSTRACT

The present invention relates to methods for screening and identifying compounds which block chloride channels of the CIC family. In addition, the present invention relates to the use of chloride channel blockers of the CIC family for the treatment, prevention or alleviation of osteoclast related bone diseases.

The present invention relates to a method for screening compounds for activity in treating an osteoclast related bone disease.

### BACKGROUND ART

Cells are protected from their external environment by a cell membrane. In order to communicate with the outside world, cells have developed transmembrane proteins, which bind external molecules to activate intracellular signal transduction mechanisms, or transport solutes in or out of the cell. Ion channels are transmembrane proteins, which catalyse the transport of inorganic ions across cell membranes.

The ion channels participate in processes as diverse as generating and timing of action potentials, synaptic transmission, secretion of hormones, contraction of muscles, signal transduction, etc. Ion channels can be divided according to the ions they conduct, i.e. K⁺ channels, Na⁺ channels and CI⁻ channels. CI⁻ channels are probably found in every cell, from bacteria to mammals. Their physiological tasks range from cell volume regulation to stabilization of the membrane potential, transepithelial or transcellular transport and acidification of intracellular organelles. These different functions require the presence of many distinct chloride channels, which are differentially expressed and regulated by various stimuli.

### Osteoporosis and CI⁻ Channels

A healthy bone results from a balance between ongoing bone formation and resorption processes. Bone formation is dependent on osteoblasts activity while osteoclasts are involved in bone resorption.

It is well known that the pathogenesis of osteoporosis can result from an imbalance between bone formation and resorption as a result of dominant osteoclasts activity.

Osteoclasts attach bone whereby a bone resorption compartment is generated as a cavity between the osteoclast and the bone. This compartment is tightly sealed and isolated from the pericellular compartment. Upon bone attachment the osteoclast plasma membrane develops into a ruffled membrane which delimits the bone resorption compartment.

The bone resorption process mediated by osteoclasts is highly dependent on acidification of the bone resorption compartment. A H⁺-ATPase transports protons from osteoclast cytosol across the ruffled membrane into the bone resorption compartment. Cl⁻ ions from osteoclast cytosol passively follow the protons through anion channels in the ruffled membrane to the bone resorption compartment. This coordinated transport of protons and CI⁻ ions (generating HCI) acidifies the bone resorption compartment resulting in dissolution of the mineral bone components and consequent exposure of the protein part of the action of osteoclast proteases.

Schlesinger, P. H. et al (J. Biol. Chem (1997) 272, 18636-18643) disclose that an anion channel expressed in the osteoclast is related to the bovine p64 CI⁻ channel, but the molecular structure of the ruffled membrane CI⁻ channel is not known.

WO 00/24707 discloses compounds useful as chloride channel blockers.

However, there is a strong interest in the provision of more effective and selective compounds with fewer side effects for the treatment of patients with an osteoclast related bone disease, such as osteoporosis.

### SUMMARY OF THE INVENTION

According to the invention it has now been found that the chloride channels of the CIC family are involved in the resorption of bone. Therefore, chloride channels of the CIC family, such as CIC-3, CIC-6, and CIC-7 constitute molecular targets for the treatment of an osteoclast related bone disease, such as osteoporosis.

Thus, in its first aspect, the invention relates to a method for screening a chemical compound for activity in the treatment, prevention or alleviation of an osteoclast related bone disease in a subject.

In its second aspect, the invention relates to a drug development method.

In its third aspect, the invention relates to the use of a compound identified as a blocker of a chloride channel of the CIC family by said screening method in the treatment, prevention or alleviation of an osteoclast related bone disease in a subject.

In its fourth aspect, the invention relates to the use of a blocker of a chloride channel of the CIC family in the treatment, prevention or alleviation of an osteoclast related bone disease in a subject.

Other objects of the invention will be apparent to the person skilled in the art from the following detailed description and examples.

### DETAILED DISCLOSURE OF THE INVENTION

In its first aspect, the invention provides a method for screening a chemical compound for activity in the treatment, prevention or alleviation of an osteoclast related bone disease in a subject, which method comprises the following steps:

providing a test cell comprising one or more chloride channels of the CIC family;

subjecting the test cell to the action of the chemical compound; and

measuring the ability of the compound to block the selected chloride channels.

In a second aspect, the invention provides a drug development method, which comprises the identification of a compound by the method as described above.

In a further aspect, the invention provides the use of a compound identified as a blocker of a chloride channel of the CIC family by the method as described above or a pharmaceutically acceptable salt or a prodrug thereof for the manufacture of a medicament for the treatment, prevention or alleviation of an osteoclast related bone disease in a subject.

In a still further aspect, the invention provides a method for the treatment, prevention, or alleviation of an osteoclast related bone disease in a subject comprising administering to said subject a therapeutically effective amount of a compound identified as a blocker of a chloride channel of the CIC family by the method as described above or a pharmaceutically acceptable salt or a prodrug thereof.

In a further aspect, the invention provides the use of a blocker of a chloride channel of the CIC family or a pharmaceutically acceptable salt or a prodrug thereof for the manufacture of a medicament for the treatment, prevention or alleviation of an osteoclast related bone disease in a subject.

In a still further aspect, the invention provides a method for the treatment, prevention, or alleviation of an osteoclast related bone disease in a subject comprising administering to said subject a therapeutically effective amount of a blocker of a chloride channel of the CIC family or a pharmaceutically acceptable salt or a prodrug thereof.

In one embodiment, the test cell comprises one or more chloride channels selected from the group consisting of CIC-3, CIC-6, CIC-7, and functional analogues thereof. In a special embodiment, the test cell comprises one or more chloride channels selected from the group consisting of CIC-3, CIC-6, and CIC-7.

In a further embodiment, the osteoclast related bone disease is osteoporosis, osteolytic cancer invation, osteopetrosis, or Paget's disease of bone.

In a still further embodiment, the blocker of a chloride channel of the CIC family is a blocker of a chloride channel selected from the group consisting of CIC-3, CIC-6, and CIC-7.

In a special embodiment, the blocker of a chloride channel of the CIC family is not a blocker of a chloride channel selected from the group consisting of CIC-1, CIC-2, CIC-4, CIC-5, CIC-Ka, and CIC-Kb.

In the context of this invention, the term “osteoclast related bone disease covers any deviation of bone resorption related to osteoclast associated diseases or disorders, such as osteoporosis, osteolytic cancer invation, osteopetrosis, or Paget's disease of. bone.

The subject to be treated according to this invention is a living body, preferably a mammal, most preferably a human, in need for such treatment.

In the context of this invention, the chloride channels of the CIC family are the chloride channels having the signature in the P1 region as described by Fahlke, C, in Kidney Int (2000) 57(3):780-6.

In the context of this invention, CIC-3 is the chloride channel of the CIC family as described by Borsani et al (Genomics (1995) 27(1) 131-141), and with GenBank Acc. No

In the context of this invention, CIC-6 is the chloride channel of the CIC family as described by Brandt, S. et al (FEBS lett. (1995) 377 15-20), and with GenBank Acc. No AF209724.

In the context of this invention, CIC-7 is the chloride channel of the CIC family as described by Brandt, S. et al (FEBS lett. (19985) 377 15-20) and with GenBank Acc. No AF224741.

Functional analogues of the chloride channels CIC-3, CIC-6, and CIC-7 are chloride channels having substantially equivalent activity as compared to the unmodified chloride channel. Such analogues include splice variants, isoforms, homologues from other species.

In the test cells, the chloride channels may exist as dimer forms, either homodimers or heterodimers, such as a heterodimer of CIC-3 and CIC-6.

In the context of this invention, a blocker of a chloride channel of the CIC family, such as CIC-3, CIC-6, and CIC-7, is a compound that blocks said chloride channel.

The ability of a compound to block a specific chloride channel, such as CIC-3, CIC-6, or CIC-7, can be measured as described in the method of example 4.

In one embodiment, the ability of a blocker of a chloride channel of the CIC family to block the CIC-3 shows an IC₅₀ value less than 100 μM, preferably less than 10 μM, and more preferably less than 1 μM.

In a further embodiment, the ability of a blocker of a chloride channel of the CIC family to block the CIC-6 shows an IC₅₀ value less than 100 μM, preferably less than 10 μM, and more preferably less than 1 μM.

In a still further embodiment, the ability of a blocker of a chloride channel of the CIC family to block the CIC-7 shows an IC₅₀ value less than 100 μM, preferably less than 10 μM, and more preferably less than 1 μM.

In a further embodiment, the ability of a blocker of a chloride channel of the CIC family to block CIC-1, CIC-2, CIC-4, CIC-5, CIC-Ka, and CIC-Kb shows an IC₅₀ value higher than 1 μM, preferably higher than 10 μM, and more preferably higher than 100 μM.

The chloride channel may or may not be endogenous to the test cell to be used in the method of screening, i.e. be a chloride channel naturally occurring In the cell.

Preferably, the chloride channel may be exogenous to the cell In question, and may in particular be introduced by recombinant DNA technology, such as transfection or infection. Such cells include human embryonic kidney (HEK) cells, in particular HEK 293 cells, Chinese hamster ovary (CHO) cells, Xenopus laevis oocytes, or any other cell lines capable to chloride channels.

Examples of pharmaceutically acceptable addition salts of the compounds of the invention include inorganic and organic acid addition salts such as the hydrochloride, hydrobromide, phosphate, nitrate, perchlorate, sulfate, citrate, lactate, tartrate, maleate, fumarate, mandelate, benzoate, ascorbate, cinnamate, benzenesulfonate, methanesulfonate, stearate, succinate, glutamate, glycollate, toluene-p-sulphonate, formate, malonate, naphthalene-2-sulphonate, salicylate and the acetate. Such salts are formed by procedures well known in the art.

Other acids such as oxalic acid, while not in themselves pharmaceutically acceptable, may be useful in the preparation of salts useful as intermediates in obtaining compounds of the invention and their pharmaceutically acceptable acid addition salts.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts, such as the sodium salt of a chemical compound of the invention containing a carboxy group.

### Prodrugs

The substance used according to the invention may be administered as such or in the form of a suitable prodrug thereof. The term “prodrug” denotes a bioreversible derivative of the drug, the bioreversible derivative being therapeutically substantially inactive per se but being able to convert in the body to the active substance by an enzymatic or non-enzymatic process.

Thus, examples of suitable prodrugs of the substances used according to the invention include compounds obtained by suitable bioreversible derivatization of one or more reactive or derivatizable groups of the parent substance to result in a bioreversible derivative. The derivatization may be performed to obtain a higher bioavailability of the active substance, to stabilize an otherwise unstable active substance, to increase the lipophilicity of the substance administered, etc.

Examples of types of substances which may advantageously be administered in the form of prodrugs are carboxylic acids, other acidic groups and amines, which may be rendered more lipophilic by suitable bioreversible derivatization. As examples of suitable groups may be mentioned bioreversible esters or bioreversible amides. Amino acids are typical examples of substances which, in their unmodified form, may have a low absorption upon administration. Suitable prodrug derivatives of amino acids will be one or both of the above-mentioned types of bioreversible derivatives.

### Method of Screening

Screening for the ability of a compound of block one or more chloride channels from the CIC family can be performed by a multitude of techniques well known in the art.

Examples of available screening methods are conventional electrophysiological methods such as patch-clamp techniques, or conventional spectroscopic methods such as FLIPR assay (Fluorescence Image Plate Reader; available from Molecular Devices), or VIPR (voltage ion probe reader, available from Aurora).

These methods generally comprise monitoring the membrane potential of the chloride channel containing cell in order to identify changes in the membrane potential caused by the action of the compound of the invention.

Other screening methods include ligand binding assays, Cl-flux based assays and oocyte voltage clamp after expression of CIC cDNA in an appropiate heterologous system.

### Pharmaceutical Compositions

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suit the desired therapy. Preferred routes of administration include oral administration, in particular in tablet, in capsule, in daage, in powder, or in liquid form, and parenteral administration, in particular cutaneous, subcutaneous, intramuscular, or intravenous injection. The pharmaceutical composition may be prepared by the skilled person using standard and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

The following examples will illustrate the invention further, however, they are not to be construed as limiting.

### EXAMPLES

### Example 1

### Identification of CIC Channels of Osteoclasts

CIC-3 (GenBank Acc. No AF029348), CIC-6 (GenBank Acc. No AF209724), and CIC-7 (GenBank Acc. No AF224741), channels were identified in human osteoclasts (developed from macrophages, OsteoPro) using a RT-PCR based strategy as described below.

Methods

Purification of total RNA from osteoclasts (using RNeasy kit, Qiagen; or standard methods such as described in Ausubel, F M, Brent, R, Kingston, R E, Moore, D D, Seidman, J G, Smith, J A and Struhl, K eds. (2000) Current protocols in Molecular Biology Vol 1, Wiley & Sons)

Sequence specific RT-PCR using PCR primers specific for the different subtypes of CI⁻ channels of the CIC, CICA and CLIC families.

RT mix

3 μg total RNA or 30-150 ng mRNA

2,5 μM oligo dT final concentration

Denaturate 10 min at 70° C., place then at ice.

Add:

1 mM dNTP final concentration (Pharmacia)

5 mM MgCl₂ final concentration (Gibco BRL)

1X PCR buffer without MgCl₂(Gibco BRL)

20 units Rnase inhibitor (Perkin Elmer)

H₂O to 20 μl final volumen

Anneal 10 min at room temperature

50 units RT Mulv (Perkin Elmer)

Incubate 1 hour at 42° C. and 15 min 70° C.

RT-PCR

1/10 of the RT mix is used in the RT-PCR reaction

2 ∞l RT mix

250 μM dNTP final concentration

0.5 μM sense and antisense primer final concentration

1.5 mM MgCl final concentration (Gibco BRL)

1X PCR buffer without MgCl₂ (Gibco BRL)

2.5 U Taq polymerase (Gibco BRL)

H₂O to 20 μl final volumen

Using the following sequence specific primers:

| | | |
|---|---|---|
| Speci- | Sense | Posi- |

| | | |
|---|---|---|
| ficity | Antisense | tion |

| | | | |
|---|---|---|---|
| CIC-3 | gcagaattaatcataggccaagca | SEQ ID NO. 1 | 580 |
| | atactttggaaagaggtaggaaaa | SEQ ID NO. 2 | 909 |
| CIC-6 | ctcctgggctttaacttgaccttc | SEQ ID NO. 3 | 397 |
| | aaactggatcttccgtaaggagat | SEQ ID NO. 4 | 681 |
| CIC-7 | ggtgtctgccggggtcttcatcc | SEQ ID NO. 5 | 1524 |
| | gagccgggcaggctgggtgtc | SEQ ID NO. 6 | 2003 |

PCR profile

94° C. 2 min. 94° C. 1 min, 60/66° C. 1 min, 72° C. 1 min repeated 29 times. 72° C. 5 min. 4° C. for ever.

### Northern Blot Using Standard Methods

Results obtained by RT-PCR may be validated by northern blot. 10 μg human osteoclast total RNA is blotted on hybond membranes (Amersham Pharmacia Biotech) and prehybridized for 30 min at 65° C. in ExpressHyb (Clontech). CIC-3, CIC-6 and CIC-7 specific P³² labeled DNA probes are generated using random priming (Amersham Pharmacia Biotech) and hybridized to the blotted membranes for 16 hours at 65° C. in ExpressHyb. The hybridized membrane is washed and analyzed by auto adiography.

### Example 2

### Expression of CIC-3, CIC-6, and CIC-7 in HEK293 Cells

The CIC-3, CIC-6, and CIC-7 were recombinant expressed in HEK293 cells using standard methods based on lipofectamine transfection (Life Technologies).

Briefly, CIC-3, CIC-6, and CIC-7 was excised from pCRII-TOPO (Invitrogen), and subcloned into a mammalian/oocyte expression vector generating expression vectors, named pZOOM_CIC3, pZOOM_CIC6, and pZOOM_CIC7.

HEK293 tissue culture cells were grown in DMEM (Dulbecco's Modified Eagle Medium) supplemented with 10% FCS (foetal calf serum) at 37° C. in 5% CO₂. One day prior to transfection, 10⁶ cells were plated in a cell culture T25 flask. The following day, cells were transfected using lipofection (20 μL Lipofectamin™, Life Technologies, with 2.5 μg of the pZOOM_CIC3, pZOOM_CIC6, and pZOOM_CIC7 expression vectors respectively in a total volume of 540 μL).

The lipofection mixture was overlaid on the cells and incubated at 37° C. for 5 hours. The cells were then rinsed with regular media and grown for 72 hours in DMEM, 10% FCS at 37° C. in 5% CO₂.

72 hours post transfection, cells transfected with pZOOM were selected in media supplemented with 0.5 μg/ml G418. Single clones were picked and propagated in selection media until sufficient cells for freezing were available. Hereafter the cells were cultured in regular medium without selection agent.

Expression of functional CIC-3, CIC-6, and CIC-7 channels was verified by patch-clamp measurements.

### Example 3

### Patch Clamp Screening for Compounds

The following method can be used for screening compounds for chloride channel blocking activity.

Experiments are carried out on one of several patch-clamp set-ups. Cells plated on coverslips are placed in a 15 μl perfusion chamber (flowrate˜1 ml/min) mounted on an IMT-2 microscope equipped with Nomarski or Hoffmann optics. The micros-copes are placed on vibration-free tables in grounded Faraday cages. All experiments are performed at room temperature (20-22° C.). EPC-9 patch-clamp amplifiers (HEKA-electronics, Lambrect, Germany) are connected to Macintosh computers via ITC16 interfaces. Data are stored directly on the harddisk and analysed by the IGOR software (WaveMetrics, Lake Oswega, USA).

The whole-cell configuration of the patch clamp technique is applied. The tip of a borosilicate pipette (resistance 2-4 MΩ) is gently (remote control system) placed on the cell membrane. Light suction results in a giga seal (pipette resistance increases to more than 1 GΩ) and the cell membrane is then ruptured by more powerful suction. Cell capacitance is electronically compensated and the resistance between the pipette and the cell interior (the series resistance, Rs) is measured and compensated for. Usually the cell capacitance ranges from 5 to 20 pF (depending on cell size) and the series resistance is in the range 3 to 6Ω. Rs- as well as capacitance compensation are updated during the experiments (before each stimulus).

All experiments with drifting Rs-values are discharged. Leak-subtractions are not performed.

The extracellular (bath) solution contains: 144 mM KCI, 2 mM CaCl₂, 1 mM MgCl₂, 10 mM HEPES (pH=7.4). Test compounds are dissolved in DMSO from stock solution and then diluted to a final concentration of about 10 μM in the extracellular solution. The concentration of CaCl2 is 7.6 mM and that of MgCl₂ is 1.2 mM to give calculated free concentrations of 300 nM and 1 mM, respectively.

Quantification

After establishment of the whole-cell configuration, voltage-ramps (usually −100 to +100 mV) are applied to the cell every 5 sec. A stable baseline current is obtained within a period of 100-300 seconds, and the compounds are then added by changing to an extracellular solution containing the compound to be tested. Very little endogen current is activated under these circumstances in native HEK293 cells.

### Example 4

### Northern Blot for Cic-type Channels

Samples of 5 μg total RNA from human fetal brain (Stratagene), 10 μg total RNA from human osteoclast cultures differentiated in the presence of RANKL for one week and 10 μg total RNA from human osteoclast cultures differentiated in the presence of RANKL for two weeks were separated on a denaturing formaldehyde gel, photographed and transferred to Hybond N membranes (Amersham) using capillary blotting.

Three membranes containing identical amounts of RNA were produced and probed with 32P-labeled PCR-fragments (Rediprime II random labelling kit, Amersham) for human CIC-7 (bases 1524-2026), human CIC-6 (bases 1171-1624) or human CIC-3 (bases 242-651), respectively. Prehybridisation and hybridisation were performed with Ambion's Ultrahyb hybridisation buffer at 42° C. for 30 min and overnight, respectively, followed by two short washes with 2×SSC/0.1% SDS and two 15 min washes with 0.1×SSC/0.1% SDS (20×SSC: 3M NaCl, 0.3 M sodium citrate, pH 7).

Membranes were wrapped in plastic foil and exposed on Kodak Biomax ML film for up to three weeks.

An approximately 4 kb transcript of human CIC-7 could be detected in brain and both human osteoclast preparations. The strongest signal was observed in the most differentiated osteoclasts (two weeks differentiation in the presence of RANKL). A weak 6.2 kb message of human CIC-6 could be observed in human fetal brain, but not in human osteoclasts, even after prolonged exposure. Human CIC-3 was not detectable in either. sample.

The size of CIC-7 and CIC-6 transcripts correspond very well to those described in: Brandt and Jentsch (1995) FEBS Letters 377, 15-20 (4.2 kb and 6 kb, respectively).

Thus, CIC-7 is the only CIC-type channel among the three tested detectable in human osteoclasts. Furthermore, the mRNA seems to be upregulated during osteoclast differentiation, indicating the relevance of this channel in osteoclast physiology. CIC-6 and CIC-3, however, do not seem to be significantly expressed in osteoclasts, but can be detected by the more sensitive RT-PCR method.

1-8. Cancelled.

9. A method for screening a chemical compound for activity in the treatment, prevention or alleviation of an osteoclast related bone disease in a subject, which method comprises the following steps:
* providing a test cell comprising one or more chloride channels of the CIC family;
subjecting the test cell to the action of the channel compound; and
measuring the ability of the compound to block the selected chloride channels.

10. The method according claim 9, wherein the test cell comprises one or more chloride channels selected from the group consisting of CIC-3, CIC-6 and CIC-7 and functional analogues thereof having substantially equivalent activity to the native forms.

11. The method according to any one of claims 9 or 10, wherein the osteoclast related bone disease is osteoporosis, osteolytic cancer invation, osteopetrosis, or Paget's disease of bone.

12. A drug development method, which comprises the identification of a compound by the method according to any one of the claims 9 to 11.

13. The use of a compound identified as a blocker of a chloride channel of the CIC family by the method according to any one ofthe claims 9 to 11 or a pharmaceutically acceptable salt or a product thereof for the manufacture of a medicament for the treatment, prevention or alleviation of an osteoclast related bone disease in a subject.

14. A method for the treatment, prevention, or alleviation of an osteoclast related bone disease in a subject comprising administering to said subject a therapeutically effective. amount of a compound identified as a blocker or a chloride channel of the CIC family by the method according to any one of the claims 9 to 11 or a pharmaceutically acceptable salt or a prodrug thereof.

15. The use of a blocker of a chloride channel of the ClC family or a pharmaceutically acceptable salt or a prodrug thereof for the manufacture of a medicament for the treatment, prevention or alleviation of an osteoclast related bone disease in a subject.

16. The use of claim 15, wherein the said blocker is a blocker of one or more channels C1C-3, ClC-6 and ClC-7 but does not block at least one of ClC-l, ClC-2, CIC-4, CIC-5, ClC-Ka and ClC-Kb.

17. A method for the treatment, prevention, or alleviation of an osteoclast related bone disease in a subject comprising administering to said subject a therapeutically effective amount of a blocker of a chloride channel of the ClC family or a pharmaceutically acceptable salt or a prpdrug thereof.


SEQUENCE LISTING (6 sequences)

SEQ ID NO: 1 - DNA, 24 bp, Homo sapiens
gcagaattaatcataggccaagca

SEQ ID NO: 2 - DNA, 24 bp, Homo sapiens
atactttggaaagaggtaggaaaa

SEQ ID NO: 3 - DNA, 24 bp, Homo sapiens
ctcctgggctttaacttgaccttc

SEQ ID NO: 4 - DNA, 24 bp, Homo sapiens
aaactggatcttccgtaaggagat

SEQ ID NO: 5 - DNA, 23 bp, Homo sapiens
ggtgtctgccggggtcttcatcc

SEQ ID NO: 6 - DNA, 21 bp, Homo sapiens
gagccgggcaggctgggtgtc